(19) United States
(12) Patent Application Publication
(10) Pub. No.: US 2026/0320367 A1
(43) Pub. Date: Oct. 15, 2026

(54) SYSTEMS AND METHODS TO PROCESS GENERATIVE OR DYNAMIC VIDEO AND IMAGES TO TARGET HEALTHY NEURAL PATHWAYS

(71) Applicant: Dandelion Science Corp., New York, NY (US)

(72) Inventors: Gopal K. CHOPRA, New York, NY (US); Ajay VERMA, Boston, MA (US); Adam HANINA, Vandoeuvres (CH); David KLEIN, Los Altos, CA (US); Jesse LIVEZEY, Jersey City, NJ (US); Dan NEMRODOV, Jersey City, NJ (US)

(21) Appl. No.: 19/482,010

(22) PCT Filed: May 9, 2024
(86) PCT No.: PCT/US2024/028659

§ 371 Date: Nov. 6, 2025

Related U.S. Application Data
(60) Provisional application No. 63/501,334, filed on May 10, 2023.

(51) Int. Cl. G06N 3/063

(52) U.S. Cl. CPC G06N 3/063

(57) ABSTRACT

Systems and methods are disclosed for modulating neural pathways to improve functional ability in a subject. The systems and methods may contain steps, including: receiving a first dataset associated with activity of one or more neural pathways of the subject; receiving a second dataset comprising at least one of at least one task performed by the subject, at least one symptom, and at least one medical condition of the subject; determining one or more abnormal neural pathways based on the first dataset and the second dataset; determining from the second dataset, one or more healthy neural pathways associated with the one or more abnormal neural pathways based on one or more criteria; and determining, based on the healthy neural pathways and abnormal neural pathways, and using a non-invasive stimulatory device, a stimulus input focused or differentiated to the healthy neural pathways.

### CROSS-REFERENCE TO RELATED APPLICATIONS

This application is a U.S. National Stage filing under 35 U.S.C. § 371 of International Application No. PCT/US2024/028659, filed May 9, 2024, which claims priority to U.S. Provisional Application No. 63/501,334 filed May 10, 2023, the entireties of which are incorporated herein by reference.

### TECHNICAL FIELD

The present disclosure relates generally to the field of neuroscience and neuroengineering and, more specifically, to systems and methods for performing neural circuit modulation to restore, remap or amplify functional ability in individuals with disrupted neural pathways.

### BACKGROUND

The human visual system relies on complex neural networks to process visual information, enabling recognition and localization of objects. Disruption of these pathways (e.g., sensory pathways due to retinal or optic nerve diseases) may lead to functional impairments despite intact neural and cortical networks directly in the functional circuit or in parallel. Current therapeutic approaches often involve invasive procedures or rely on pharmaceutical interventions, limiting accessibility and efficacy. Moreover, traditional methods might not adequately address the dynamic nature of neural plasticity, which offers opportunities for functional recovery through targeted stimulation of healthy neural circuits.

The present disclosure is accordingly directed to non-invasive, innovative approaches that leverage advancements in neuroscience and technology to modulate neural circuitry and restore functional ability in individuals with disrupted or dysfunctional pathways. The background description provided herein is for the purpose of generally presenting context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, systems and methods are described for restoring functional ability in individuals with disrupted or dysfunctional neural pathways.

In one aspect, a computer-implemented method for modulating neural pathways to improve functional ability in a subject is provided. The computer-implemented method may include: receiving, at a computing device, a first dataset associated with activity of one or more neural pathways of the subject; receiving, at the computing device, a second dataset comprising at least one of at least one task performed by the subject, at least one symptom, and at least one medical condition of the subject; determining, using a processor associated with the computing device, one or more abnormal neural pathways based on the first dataset and the second dataset; determining, using the processor and from the second dataset, one or more healthy neural pathways associated with the one or more abnormal neural pathways based on one or more criteria; and determining, based on the healthy neural pathways and abnormal neural pathways, and using a non-invasive stimulatory device, a stimulus input to the healthy neural pathways.

In another aspect, a system for modulating neural pathways to improve functional ability in a subject is provided. The system may include: one or more processors; one or more computer readable media storing instructions that are executable by the one or more processors to perform operations for: receiving, at a computing device, a first dataset associated with activity of one or more neural pathways of the subject; receiving, at the computing device, a second dataset comprising at least one of at least one task performed by the subject, at least one symptom, and at least one medical condition of the subject; determining, using a processor associated with the computing device, one or more abnormal neural pathways based on the first dataset and the second dataset; determining, using the processor and from the second dataset, one or more healthy neural pathways associated with the one or more abnormal neural pathways based on one or more criteria; and determining, based on the healthy neural pathways and abnormal neural pathways, and using a non-invasive or invasive stimulatory device, a stimulus input to the healthy neural pathways.

In yet another aspect, a non-transitory computer-readable medium storing computer-executable instructions is provided. The non-transitory computer-readable medium stores computer-executable instructions which, when executed by a system, may cause the system to perform operations including: receiving, at a computing device, a first dataset associated with activity of one or more neural pathways of the subject; receiving, at the computing device, a second dataset comprising at least one of at least one task performed by the subject, at least one symptom, and at least one medical condition of the subject; determining, using a processor associated with the computing device, one or more abnormal neural pathways based on the first dataset and the second dataset; determining, using the processor and from the second dataset, one or more healthy neural pathways associated with the one or more abnormal neural pathways based on one or more criteria; and determining, based on the healthy neural pathways and abnormal neural pathways, and using a non-invasive stimulatory device, a stimulus input to the healthy neural pathways or across healthy and diseased pathways with a differential impact on healthy pathways.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate several embodiments and together with the description, serve to explain the principles of the disclosure.

FIG. 1A depicts an exemplary computer system for executing the methods described herein.

FIG. 1B depicts an exemplary software platform for executing the methods described herein.

FIG. 2 depicts an exemplary workflow for retraining neural centers in general clinical use, according to one or more embodiments of the present disclosure.

FIG. 3 depicts an exemplary workflow for training a machine learning model that may be leveraged in the application of non-invasive neurostimulation for retraining visual processing pathways in general clinical use, according to one or more embodiments of the present disclosure.

FIG. 4 depicts an example computing system, according to one or more embodiments of the present disclosure.

### DETAILED DESCRIPTION OF EMBODIMENTS

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. Relative terms, such as “about,” “approximately,” “substantially,” and “generally,” are used to indicate a possible variation of ±10% of a stated or understood value. In addition, the term “between” used in describing ranges of values is intended to include the minimum and maximum values described herein. The use of the term “or” in the claims and specification is used to mean “and/or” unless explicitly indicated to refer to alternatives only or the alternatives are mutually exclusive, although the disclosure supports a definition that refers to only alternatives and “and/or.” As used herein “another” may mean at least a second or more.

As used herein, the term “user” generally encompasses any person or entity, such as a researcher and/or a care provider (e.g., a doctor, etc.), that may desire information, resolution of an issue, or engage in any other type of interaction with a provider of the systems and methods described herein (e.g., via an application interface resident on their electronic device, etc.). The term “electronic application” or “application” may be used interchangeably with other terms like “program,” or the like, and generally encompasses software that is configured to interact with, modify, override, supplement, or operate in conjunction with other software.

Disruptions in neural pathways, particularly within the visual system, pose significant challenges to restoring functional ability in individuals affected by various conditions. For example, retinal diseases and optic nerve damage can be debilitating ailments, often resulting in severe impairments in vision. Despite the intricate network of neural connections and cortical processing centers in the brain remaining intact, the loss of functionality in these pathways may profoundly impact an individual's ability to perceive and interpret visual stimuli. This impairment may not only affect everyday activities but may also diminish overall quality of life. For instance, with diseases such as macular degeneration or loss of limb, the cortical circuits that would process visual or touch information may be intact. However the afferent neural circuitry is dysfunctional or partially absent. For example, a subject may have a scotoma due to central visual field loss (CFL) with macular degeneration, which is a partial loss of vision or blind spot in an otherwise functioning visual cortex. The subject might have functioning vision around the scotoma, but the presence of the blind spot in the middle of the visual field might nonetheless inhibit object recognition, object manipulation, and other activities of daily living (ADL). Around the scotoma there may be the immediate surrounding visual region and the larger surrounding visual region.

Conventional attempts at addressing these issues have primarily relied on invasive procedures or pharmaceutical interventions. For instance, surgical interventions, while effective in some cases, carry inherent risks and may not be suitable for all subjects. Pharmaceutical treatments, on the other hand, often provide symptomatic relief but might not address the underlying neural dysfunction.

Furthermore, conventional techniques might not fully capitalize on the dynamic nature of neural plasticity, which offers opportunities for functional recovery through targeted stimulation of healthy neural circuits. Static stimulation protocols may overlook individual variations in neural responsiveness and fail to adapt to changes in neural activity over time. Additionally, the lack of personalization and neural stratification in conventional approaches may result in suboptimal outcomes for subjects with diverse neurological conditions.

To address the foregoing issues, the present disclosure contemplates novel techniques in neural circuit modulation, focusing on non-invasive and personalized stimulation methods tailored to individual subject characteristics and responsiveness. By integrating machine learning algorithms and sophisticated data analysis techniques, the concepts described herein may optimize stimulation parameters based on real-time subject feedback and neural activity monitoring. This dynamic approach enables the system to adapt and refine stimulation patterns over time, thereby maximizing efficacy and subject outcomes.

More particularly to the foregoing, when certain neural pathways are dysfunctional or impaired due to neurological conditions, such as retinal or optic nerve disease in the case of visual impairments, the brain may rely on alternative pathways to compensate for the loss of function. These alternative pathways, often referred to as “healthy” or “intact” or “normal” neural circuits, may still be capable of processing sensory information or generating motor commands. Neurostimulation techniques may be used to activate these healthy neural pathways, essentially rerouting neural signals to bypass the defective pathways. By stimulating regions of the brain associated with intact sensory processing, motor control, or cognitive function, neural responses may be invoked that contribute to the desired functional outcomes. Through repeated stimulation and training, the brain may undergo a process of functional reorganization or neuroplasticity. This adaptive mechanism may allow the brain to optimize its neural connections and utilize available neural resources for functional recovery and thus compensate for deficits in dysfunctional pathways. Ultimately, the goal of targeting healthy neural pathways is to enable the brain to achieve the same, or at least partial, functional outcomes or tasks that would normally be mediated by the defective pathways. Whether it's restoring visual perception, motor coordination, cognitive function, or sensory processing, neurostimulation therapy may harness the brain's inherent plasticity to promote recovery and functional improvement through available healthy neural networks and circuitry. Within visual function disorders, in age-related macular degeneration, while there is a loss of photoreceptors in the retina leading to missing visual input, the retinal-cortical pathways remain intact. Training spared cortex can fill in blanks. In glaucoma, retinal ganglion cell and optic nerve damage causes visual field loss.

However, the cortex remains functional and plasticity can map new inputs. In optic neuritis (Multiple Sclerosis), an inflamed optic nerve leads to impaired signal transmission. However the cortex is undamaged and retraining can compensate. In occipital lobe stroke, direct damage to visual cortex causes blindness. But plasticity of spared cortex allows remapping of function. Hemianopia and hemineglect result in loss of visual field after stroke/trauma. Spared cortex can be trained to fill in input. Prosopagnosia causes a loss of face recognition. Spared cortex can be trained to fill in input. Amblyopia results in impaired visual development but the cortex is intact. Plasticity can allow retraining of processing. In neurodegenerative disorders, posterior cortical atrophy (a subtype of Alzheimer's), the occipital/parietal lobe dysfunction impairs visual processing. Retraining can compensate. Dementia with Lewy bodies, Alpha-synuclein pathology impairs visual processing. Spared cortex can be retrained. In Parkinson's disease, progressive visual deficits occur from dopamine loss. The spared cortex can be trained to adapt. In Huntington's disease, eventual visual cortex dysfunction occurs, however, retraining can be beneficial to visual processing while plasticity remains.

Features of the inventive concepts include the utilization of non-invasive stimulation modalities, such as visual, auditory, tactile, electrical, and/or multimodal stimuli, delivered via specialized stimulatory devices. These stimuli may be precisely calibrated to target specific healthy or healthy beyond-a-threshold neural pathways identified through comprehensive subject assessments, including brain imaging, electrophysiological recordings, and clinical evaluations. Machine learning algorithms may also play an important role in analyzing vast amounts of subject-specific data to tailor stimulation protocols to an individual's needs and responses. Moreover, the concepts described herein may integrate with existing therapeutic modalities, complementing pharmaceutical treatments and surgical interventions. Accordingly, the systems and methods described herein represent a promising avenue for restoring functional ability in individuals with disrupted neural pathways while minimizing the risks and limitations associated with invasive procedures and static treatment protocols.

In an aspect, the collective concepts presented in this disclosure offer concrete and tangible applications in neuroscience and neuroengineering. Additionally, these concepts represent improvements in computer technology by introducing innovative applications at the intersection of neuroscience and computing. For instance, the processing and analysis of complex neural data requires sophisticated computational techniques and algorithms. The concepts described herein may leverage advancements in data analysis and processing capabilities to extract meaningful insights from large-scale neural datasets, enabling personalized treatment approaches tailored to individual subject characteristics. Additionally, by tailoring stimulation protocols to individual characteristics, including neural activity patterns and responsiveness, the system(s) and techniques described herein ensure optimal therapeutic outcomes for each subject. Additionally, the concepts described herein address a significant and specific problem in the field of neurology: the restoration of functional ability in individuals with disrupted neural pathways. Conditions such as retinal diseases and optic nerve damage have tangible and debilitating effects on subjects' vision and quality of life. By offering innovative solutions that directly target these challenges, the inventive concepts provide practical and meaningful benefits to subjects and clinicians. More particularly, by systematically targeting and stimulating healthy neural pathways, the effect of abnormally firing pathways may be diminished and/or functional pathways to be potentiated. Pain and/or symptoms may be alleviated, and function may be restored, depending on the medical condition, as the result of prioritization of functional information over dysfunctional information, and/or a gating/interrupting effect in a circuit that is dysfunctional.

The subject matter of the present disclosure will now be described more fully hereinafter with reference to the accompanying drawings, which form a part hereof, and which show, by way of illustration, specific exemplary embodiments. An embodiment or implementation described herein as “exemplary” is not to be construed as preferred or advantageous, for example, over other embodiments or implementations; rather, it is intended to reflect or indicate that the embodiment(s) is/are “example” embodiment(s). Subject matter may be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware, or any combination thereof. The following detailed description is, therefore, not intended to be taken in a limiting sense.

Throughout the specification and claims, terms may have nuanced meanings suggested or implied in context beyond an explicitly stated meaning. Likewise, the phrase “in one embodiment” or “in some embodiments,” or “in one aspect” or “in some aspects” as used herein does not necessarily refer to the same embodiment or aspect, and the phrase “in another embodiment” or “in another aspect” as used herein does not necessarily refer to a different embodiment or aspect. It is intended, for example, that claimed subject matter include combinations of exemplary embodiments in whole or in part.

While certain embodiments are discussed herein, this is not meant to be limiting. Other use cases may include migraines, dysfunctional neuropathies, disorders of the vagal or trigeminal nerves, motor dysfunction that results in tremor, or other situations where abnormally firing afferent neurons may be denying the brain normal feedback, or crowding the brain with information in ways that may cause pain, abnormal physiologic/autonomic response like nausea and hypotension, discomfort, and/or reduced function. Additionally, certain embodiments discussed herein may ameliorate dysfunction caused by disorders of the eye, but other embodiments may utilize normal afferent functioning of the eye to treat conditions manifesting in other body systems and elsewhere in the body. As will be discussed, healthy neural circuits may be targeted that are within a predetermined proximity of abnormal neural circuits. The proximity may be of processing centers that are subcortical and/or cortical. The proximity may be at any points in the neural pathways, including within the brain itself. With phantom limb pain, for example, neural pathways of the eye may be normal, thus there is no afferent disturbance, but the processing from a non-invasive visual stimulatory device may act to interrupt or gate the signal processing of an overlapping system where an abnormal afferent associated with the missing limb is sending an errant pain signal. In this way, a normally functioning circuit may override or interrupt the abnormal dysfunctional signal to alleviate a symptom. This interruption may occur at the nuclear, ganglia, and/or cortical layer and thus may impact cortical interpretation by way of remapping or reprioritizing signals.

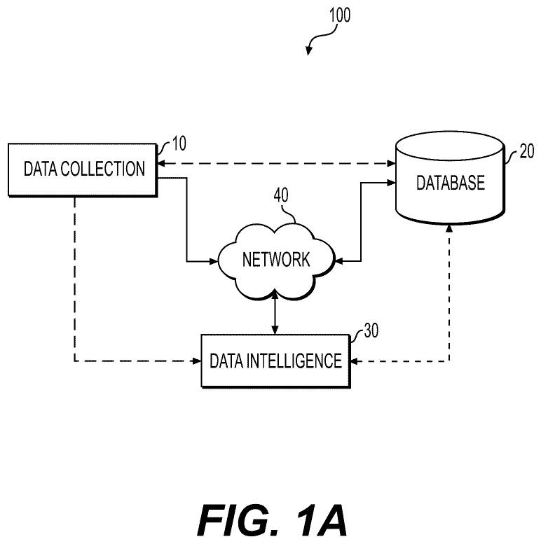

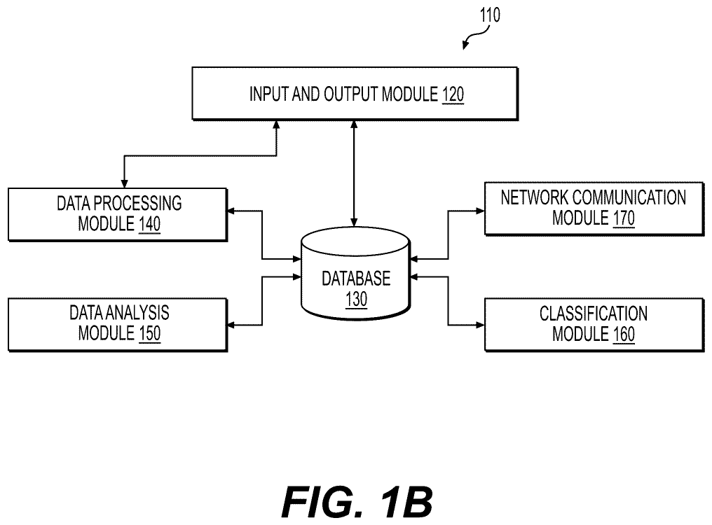

FIG. 1A depicts an exemplary system by which the methods described herein may be executed. Exemplary system 100 includes a data collection component 10, a database 20, and device data intelligence component 30, operably connected to each other via network 40. Alternatively, or additionally, one or more of the components may be connected with another component locally without reliance on network connection; e.g., through a wired connection.

As disclosed herein, data collection component 10 may include a device or machine with which electrical activity in the brain may be measured. In some embodiments, data collection component 10 may be an electroencephalograph (EEG) machine that contains, or is configured to support, one or more electrodes, amplifiers, filters, analog to digital converters, etc., by which to conduct an EEG test. Other devices such as a magnetoencephalography (MEG) machine may be used. In some aspects, data collection component 10 may be a database that receives EEG test data from one or more other sources. In other aspects, data collection component 10 may be any other brain recording device or modality that may convey information about neural activity. Consumer-grade virtual and augmented reality headsets that integrate data collection components into the headsets may be another form of device that can be utilized.

Data acquired by the data collection component 10 may be transferred to database 20 via network 40 or a direct, local or network connection. In some embodiments, the collected data may be analyzed by data intelligence component 30, via network 40 or a local or network connection. FIG. 1B depicts exemplary functional modules that may be implemented to perform tasks of data intelligence component 30.

FIG. 1B depicts an exemplary computer system 110 for using techniques discussed herein, for example for retraining visual processing pathways. Exemplary system 110 may practice the techniques discussed herein by implementing, on one or more computer devices, user input and output (I/O) module 120, memory or database 130, data processing module 140, data analysis module 150, classification module 160, network communication module 170, and any other functional modules that may be needed for carrying out a particular task (e.g., an error correction or compensation module, a data compression module, etc.). These modules may correspond to the modules of FIG. 1A. For example, database 130 may correspond to database 20, modules 140, 150, 160, and 170 may correspond to data intelligence 30, and the input aspect of module 120 may correspond to data collection 10. As disclosed herein, user I/O module 120 may further include an input sub-module, such as a keyboard, MEG, EEG, eye tracking data, and an output sub-module, such as a display (e.g., a printer, a television, a smartphone, a monitor, a virtual reality (VR) device, and/or a touchpad). In some embodiments, all functionalities may be performed by one computer system. In some embodiments, the functionalities are performed by more than one computer system. The various modules (e.g., for data processing, analysis, classification, communication, etc.) may be one or more processes executing in a distributed computing environment. For instance, in some embodiments, one or more components of the computer system 110 may be network accessible via cloud infrastructure. For example, the database 130 used to store data may be stored in one or more remote cloud servers. In this regard, the database may be one or more large storage buckets (e.g., cloud-based storage buckets such as simple storage service “S3” buckets, etc.) from which data may be retrieved on demand. As another example, data processing, analysis, and classification may be performed in cloud-based environments using services like cloud-based data processing platforms, serverless computing, cloud-based machine learning platforms, and the like.

Also disclosed herein, a particular task may be performed by implementing one or more functional modules. In particular, each of the enumerated modules itself may, in turn, include multiple sub-modules implementing one or more techniques discussed herein. For example, data processing module 140 may include a sub-module for data quality evaluation (e.g., for performing iterative refinement and validation), a sub-module for normalizing any assigned weights to ensure that the weights contribute proportionally to the overall response, a sub-module for performing interpolation or extrapolation, and the like.

In some embodiments, a user may use I/O module 120 to manipulate data that is available either on a local device or can be obtained via a network connection from a remote service device or another user device. For example, I/O module 120 may allow a user, e.g., via a keyboard, a mouse, or a touchpad, to perform data analysis via a graphical user interface (GUI). In some embodiments, a user may manipulate data via voice control. In some embodiments, user authentication may be required before a user is granted access to the data being requested. In some embodiments, user I/O module 120 may be used to manage various functional modules. For example, a user may request via user I/O module 120 input data while an existing data processing session is in process. A user may do so by selecting a menu option or type in a command discretely without interrupting the existing process. In another example, a user may utilize user I/O module 120 to set various thresholds, configure sample matching settings, and/or provide other instructions to computer system 110 that dictate how electrical signals in the brain are captured and/or monitored. As disclosed herein, a user may use any type of input to direct and control data processing and analysis via I/O module 120.

In some embodiments, system 110 further comprises a memory and/or database 130. In some embodiments, database 130 comprises a local database that may be accessed via user I/O module 120. In some embodiments, database 130 comprises a remote database that may be accessed by user I/O module 120 via network connection. In some embodiments, database 130 is a local database that stores data retrieved from another device (e.g., a user device or a server). In some embodiments, memory or database 130 may store data retrieved in real-time from internet searches. In some embodiments, database 130 may send data to and receive data from one or more of the other functional modules, including, but not limited to, a data collection module (not shown), data processing module 140, data analysis module 150, classification module 160, network communication module 170, and etc. In some embodiments, some or all real-sample data and/or synthetic sample data may be stored on database 130.

In some embodiments, database 130 may be a database local to the other functional modules. In some embodiments, database 130 may be a remote database that may be accessed by the other functional modules via wired or wireless network connection (e.g., via network communication module 170). In some embodiments, database 130 may include a local portion and a remote portion.

In some embodiments, system 110 comprises a data processing module 140. Data processing module 140 may receive the real-time data, from I/O module 120 or database 130. In some embodiments, data processing module 140 may perform standard data processing algorithms, such as one or more of noise reduction, signal enhancement, normalization, interpolation and/or extrapolation, etc. In some embodiments, data processing module 140 may be configured to process received and/or collected neural activity data associated with one or more subjects. In various embodiments, data processing module 140 may additionally create a training data set, on which one or more machine-learning models (e.g., for classification, clustering, scoring, etc.) may be trained.

In some embodiments, system 110 comprises a data analysis module 150. In some embodiments, data analysis module 150 includes identifying brain activity patterns associated with particular medical conditions, as described in connection with data processing module 140.

In some embodiments, system 110 comprises a classification module 160, which may embody a “machine-learning model” or “trained classifier.” As used herein, a “machine-learning model” or “trained classifier” generally encompasses instructions, data, and/or a model configured to receive input, and apply one or more of a weight, bias, classification, and/or analysis on the input to generate an output.

The output may include, for example, a classification of the input, an analysis based on the input, a design, process, prediction, and/or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model(s) may include deployment of one or more machine-learning techniques, such as k-nearest neighbors, linear regression, logistic regression, random forest, gradient boosted machine (GBM), deep learning, a deep neural network (e.g., recurrent neural network (RNN), convolutional neural network (CNN), Transformers) and/or any other suitable machine-learning technique. Supervised, semi-supervised, and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

Techniques discussed herein may also be implemented using multiple machine learning models, which may be executed in series and/or in parallel. For example, a first machine learning model may detect and/or interpret a neurological signal from the patient, and a second machine learning model may generate neurostimulatory imagery to present to the patient to achieve the desired result.

Neurostimulatory imagery may be generated using deep learning models. The deep learning models may have pre-trained weights or the weights may be learned from training on collected datasets which may combine visual stimulation, neural recordings, and behavioral recordings. The visual stimuli may be generated from a deep learning model that generates a group of video frames simultaneously from the stimulation parameters in a closed-loop fashion, and/or they may be generated frame-by-frame, conditioned on the changing neural data being recorded in real-time. Visual stimulation may also be generated from pre-specified visual features, e.g., gratings or white noise, or from combinations of pre-specified visual features and features generated from a deep learning model.

In an exemplary use case, a machine-learning model may be trained to analyze test data from a test subject whose specific neural activity with respect to a medical condition may be unknown and then subsequently identifying portions or characteristics of the test subject's brain that may be responsible for or may be resultant of the medical condition. In some embodiments, the one or more parameters may include a score (e.g., a binomial probability score that may be calculated based on logistic regression analysis). As disclosed herein, the binomial probability score may correspond to the likelihood of a subject having a certain medical condition, the likelihood of a portion of the subject's brain being active or inactive, the likelihood of a particular stimuli affecting a desired portion of the brain, etc. For example, a score of over a predefined threshold may indicate that a specific stimulus or sequence or set of stimuli has effectively stimulated a non-sensory region of the brain.

As disclosed herein, network communication module 170 may be used to facilitate communications between a user device, one or more databases, and any other suitable system or device through a wired or wireless network connection. Any communication protocol/device may be used, including, without limitation, a modem, an Ethernet connection, a network card (wireless or wired), an infrared communication device, a wireless communication device, and/or a chipset (such as a Bluetooth™ device, an 802.11 device, a WiFi device, a WiMax device, cellular communication facilities, etc.), a near-field communication (NFC), a Zigbee communication, a radio frequency (RF) or radio-frequency identification (RFID) communication, a PLC protocol, a 3G/4G/5G/LTE based communication, and/or the like. For example, a user device having a user interface platform for processing/analyzing tumor fraction data may communicate with another user device with the same platform, a regular user device without the same platform (e.g., a regular smartphone), a remote server, a physical device of a remote IoT local network, a wearable device, a user device communicably connected to a remote server, and etc.

Techniques disclosed herein may be used in combination with those discussed in U.S. Pat. No. 10,736,526 and U.S. App. Ser. No. 18/044,054, each of which are incorporated by reference herein in their entireties.

The functional modules described herein are provided by way of example. It will be understood that different functional modules may be combined to create different utilities. It will also be understood that additional functional modules or sub-modules may be created to implement a certain utility.

### General Clinical Use

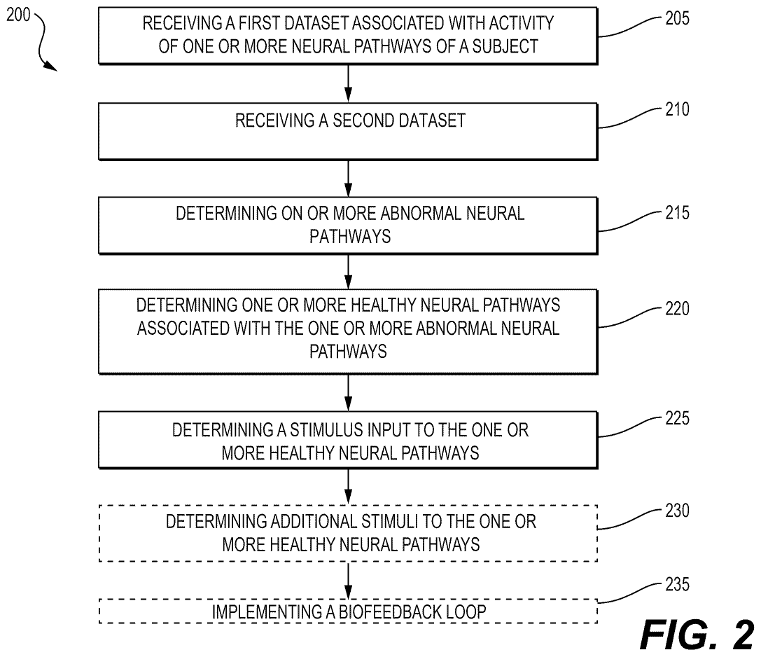

Referring now to FIG. 2, an exemplary workflow 200 is provided for utilizing the embodiments described herein to retrain neural centers in general clinical use. Aspects of the exemplary workflow 200 may be performed in accordance with some or all components described in FIGS. 1A and 1B.

At step 205, the system may receive electronic and/or magnetic data related to the activity of one or more neural circuits of a subject. This data may encompass various types of signals associated with brain activity, including electrical, magnetic, and/or blood flow/oxygenation activity. For example, electrical activity may encompass signals representing the electrical impulses generated by neurons in the brain, which may be captured using devices such as an EEG.

Magnetic activity may encompass magnetic fields generated by neural activity, which may be measured using techniques and devices like MEG. Blood flow/oxygenation activity may encompass changes in blood flow and/or oxygenation in different areas of the brain, which may indicate neural activity. Techniques such as functional magnetic resonance imaging (fMRI) may be leveraged to capture these changes. In some aspects, prior to data collection, subjects may need to undergo certain preparations depending on the imaging technique being used. For example, for EEG records, electrodes may be placed on the scalp after cleaning and preparing the skin. In fMRI studies, subjects may need to lie still within the MRI machine while data is collected. Functional near-infrared spectroscopy (fNIRS) which uses near-infrared light to measure changes in cerebral blood flow—by quantifying hemoglobin concentration changes in the brain based on optical intensity measurements—could also be leveraged.

At step 210, the system may receive a second dataset comprising data about one or more subject symptoms and/or medical conditions. More particularly, to facilitate this identification, after collection, the raw data may undergo preprocessing to extract relevant information and eliminate noise. This preprocessing may involve performing filtering, artifact removal, and/or other signal analysis techniques. The preprocessed data may then be stored for further analysis. In some aspects, data analysis may involve comparing signals across different brain regions, identifying patterns of activity, and correlating activity with specific stimuli or tasks. For instance, the comparison may involve comparing the subject's brain activity to established norms or reference data to determine deviations indicative of healthy and/or abnormal neural pathways. Based on the indication of symptoms or medical condition, one or more abnormal neural pathways may be identified. This may involve analyzing data from imaging devices or other diagnostic tools to pinpoint areas of dysfunction. In some cases, abnormal pathways may not be explicitly identified, and interventions may be automatically derived from subjects' brain activity without explicit interpretation of abnormality or pathway.

At step 210, the system may also and/or alternatively receive a second dataset associated with the performance of motor and/or cognitive tasks performed by the subject. This dataset may be obtained during the original data capture session or may be obtained at one or more later times and/or during one or more subsequent data capture sessions. In an aspect, during data capture for the second dataset, subjects may be asked to perform one or more motor and/or cognitive tasks, which may help potentiate or target healthy and/or abnormal pathways at step 215. The performance of motor and cognitive tasks serves multiple purposes in the context of non-invasive neurostimulation. For instance, performing tasks engages specific neural pathways associated with motor or cognitive function. This activation helps potentiate these pathways, making them more receptive to subsequent stimulation. Additionally, by observing the subject's response to tasks, clinicians can identify both healthy and abnormal neural pathways. Healthy pathways demonstrate appropriate responsiveness to tasks, while abnormal pathways may exhibit altered or impaired function. If abnormal pathways are not explicitly identified, automatically derived interventions may utilize data about task performance in addition to neural data.

In an aspect, motor tasks may involve physical movements or actions performed by the subject. They may range from simple actions like finger tapping or hand grasping to more complex movements like walking or reaching for objects.

Motor tasks engage motor-related neural circuits and may help assess motor function and coordination. On the other hand, cognitive tasks may involve mental processes such as attention, memory, language, and executive function. Subjects may be asked to perform tasks such as memory recall, problem-solving, or attentional focus. Cognitive tasks engage higher-order cognitive neural circuits and may provide insights into cognitive function and processing. In an aspect, the choice of tasks performed may be tailored to the subject's symptoms and medical condition. For example, subjects with visual impairments may be asked to perform tasks related to visual processing, such as object recognition or spatial navigation. In an aspect, the tasks should engage neural pathways relevant to the subject's condition. For instance, motor tasks may target regions of the brain associated with motor control, while cognitive tasks may target areas involved in visual processing or attention. Tasks should be feasible for the subject to perform safely within the clinical setting and should be appropriate for the subject's age, physical condition, and cognitive abilities. During the performance of motor and cognitive tasks, the subject's behavior and responses may be observed. This may involve assessing movement quality, accuracy, speed, or cognitive performance metrics such as reaction time or accuracy on cognitive tasks. In some aspects, disparate tasks may be combined (e.g., walking and talking, etc.), as there may be a stimulatory effect by engaging a different, yet easily activated, part of the brain to increase resting stimulatory susceptibility.

At step 220, the system may leverage the second dataset and/or first dataset to identify one or more neural pathways in the brain that are functioning normally and are suitable targets for neurostimulation. The selection of these pathways may be based on one or more criteria. For instance, healthy neural pathways selected for neurostimulation may be in close proximity to identified abnormal pathways or regions of dysfunction. Stimulating healthy pathways adjacent to abnormal areas may allow for modulation of neural activity and restoration of function. For example, in subjects with visual impairments due to retinal or optic nerve disease, healthy neural pathways associated with intact visual processing areas may be targeted to compensate for deficits in dysfunctional regions. In another aspect, selected healthy pathways should be functionally relevant to the subject's symptoms and medical condition. Stimulating pathways that contribute to the desired functional outcome may be important for achieving therapeutic benefits. Additionally or alternatively, in another aspect, the feasibility of stimulating identified pathways using non-invasive neurostimulation techniques. Factors such as accessibility, safety, and potential for therapeutic benefit may influence the selection of target sites. Additionally or alternatively, in another aspect, the selected pathways may have therapeutic potential, meaning that stimulation of these pathways is likely to result in meaningful improvements in the subject's symptoms and functional outcomes. Additionally or alternatively, in another example, the safety of neurostimulation interventions may be optimized by selecting only those pathways with a favorable safety profile and avoiding areas associated with potential adverse effects or unintended consequences. Additionally or alternatively, in yet another aspect, the individual characteristics of each subject, including their medical history, neuroanatomy, and response to previous treatments may be considered to tailor the selection of healthy pathways to the specific needs and circumstances of the subject. In some cases, healthy and abnormal pathways may not be explicitly identified; automated interventions may use data from individuals exhibiting dysfunction and optionally data from healthy individuals, to infer effective stimulus interventions.

In an aspect, one or more methods/techniques may be employed for this data identification step. For instance, advanced imaging techniques such as magnetic resonance imaging (MRI), functional MRI (fMRI), positron emission tomography (PET), or diffusion tensor imaging (DTI) may provide detailed insights into the structure and function of the brain. These imaging modalities may be utilized to visualize neural pathways and identify regions of the brain associated with healthy function. In another aspect, electrophysiological methods, such as EEG or MEG, can measure electrical activity in the brain. By recording neural signals, patterns of activity associated with healthy neural pathways may be identified. In some cases, this step may be bypassed when using an automated purely data-driven approach.

At step 225, neurostimulation techniques may be determined and/or output for application to the identified healthy neural pathways in order to modulate neural activity and promote functional improvement in subjects with neurological disorders. By targeting these pathways, neural function may be restored or enhanced to alleviate symptoms, and improve overall quality of life. Neurostimulation may be facilitated by various types of non-invasive neurostimulation. For instance, in one aspect, Transcranial Magnetic Stimulation (TMS) may be utilized to deliver magnetic pulses to specific regions of the brain, inducing electrical currents that modulate neural activity. TMS is conventionally utilized to stimulate cortical areas associated with motor function, cognition, or mood regulation. In another aspect, Transcranial Direct Current Stimulation (tDCS) may be utilized to apply low-intensity direct electrical currents to the scalp to modulate neuronal excitability. This approach may enhance or inhibit neural activity in targeted brain regions and is conventionally used for cognitive enhancement or mood regulation. In some cases, stimulation may not explicitly target identified healthy pathways but may instead strive to converge upon patterns of neural activity empirically associated with improved functional performance without human interpretation.

In an aspect, the frequency of stimulation may be varied (e.g., by, or based on output from, a trained machine learning model, as further described herein) to produce different effects on neural excitability. For instance, high-frequency stimulation may enhance synaptic plasticity and cortical excitability, while low-frequency stimulation may have inhibitory effects. In an aspect, stimulation that is delivered in specific temporal patterns, such as bursts or series of pulses, may influence the synchronization of neural activity and induce plastic changes in neural circuits. In an aspect, closed-loop systems may integrate real-time feedback from physiological signals or neural activity to adjust the parameters of stimulation dynamically.

At step 230, if a proximal healthy neural pathway is not stimulated by the first stimulus, or if the stimulus fades in effectiveness over time, additional stimuli may be determined and provided to attempt to stimulate the healthy neural pathway, or another healthy neural pathway. Stimulus of the healthy neural pathway may continue for a predetermined amount of time, based on subject feedback, and/or based on updates to the electronic subject information. For example, the electronic subject information may reflect desired retraining of neural pathways in the subject's brain, at which point the stimulus may be discontinued.

At step 235, a biofeedback loop may be incorporated into the neurostimulation process to optimize therapy by providing real-time data on neural activity, physiological responses, or behavioral changes during stimulation. This feedback mechanism may be leveraged to dynamically adjust stimulation parameters and tailor treatment interventions based on individual subject responses.

In an aspect, biofeedback data may be collected through various monitoring modalities described above, including neuroimaging, electrophysiological recordings, physiological sensors, and/or subject-reported outcomes. These data sources capture relevant information about neural activity, physiological parameters, symptomatology, and functional performance. In some aspects, the biofeedback data may be provided as input to a trained machine learning model (as further described herein) that may be configured to process the data in substantially real-time to extract meaningful insights (e.g., treatment efficacy, subject progress, etc.) into subject responses to neurostimulation. Various algorithms and analytical techniques may be employed to identify patterns, trends, and anomalies in the data to facilitate rapid decision-making (e.g., adjustment of stimulus parameters, treatment protocols, etc.) and adaptive care.

These techniques may be used to, for example, treat and/or reduce hallucinations or other symptoms experienced with Charles Bonnet syndrome or dementia by stimulating healthy brain areas. In other words, reactivating suppressed normal and functional neurocircuitry that have otherwise been cut off or isolated from normal activity and are consequently activating functions with negative consequences.

### General Model Training and Deployment

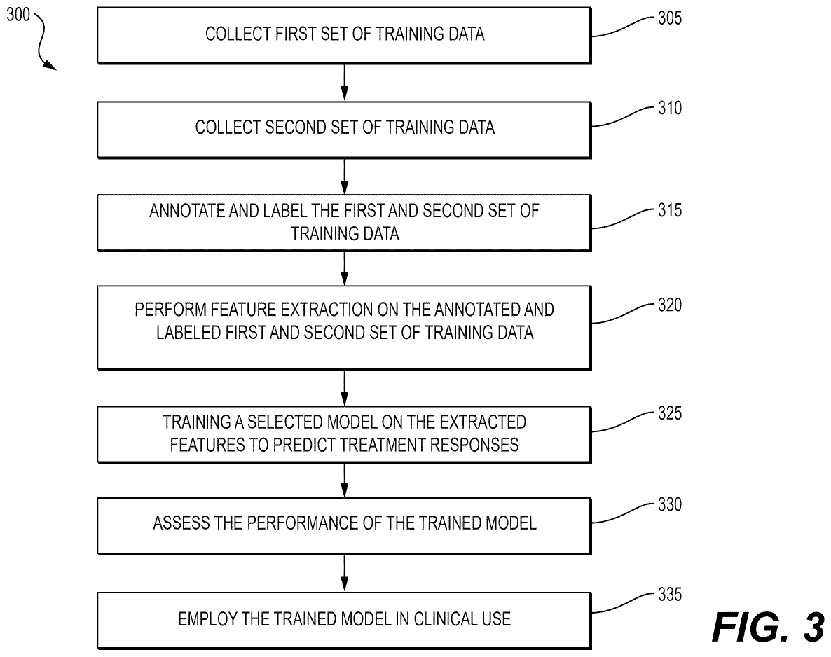

Referring now to FIG. 3, an exemplary workflow 300 is provided for training a machine learning model that may be leveraged in the application of non-invasive neurostimulation for retraining visual processing pathways. Aspects of the exemplary workflow 300 may be performed in accordance with some or all components described in FIGS. 1A and 1B, as well as some or all processes described in exemplary workflow 200.

In an aspect, machine learning algorithms may be employed to analyze electronic data obtained from subjects, including neural activity captured through imaging devices like EEG or MEG, and behavioral task performance affected by the dysfunction of interest. By processing this data, the algorithms may identify patterns and correlations specific to each subject's condition. This personalized approach allows for the customization of neurostimulation protocols tailored to the individual's neural responses and symptomatology. For example, the algorithms may determine the optimal type, intensity, and duration of stimuli based on the subject's unique neural profile.

At step 305, a first set of training data may be collected. In an aspect, the first set of training data may correspond to inputs provided by a non-invasive stimulatory device to a plurality of subjects undergoing neurostimulation treatment.

More particularly, each data point in the first set of training data corresponds to a specific stimulus presented to a subject, along with relevant metadata such as the type of stimulus, its duration, intensity, frequency, and other parameters.

At step 310, the second set of training data may be collected. In an aspect, the second set of training data may correspond to the corresponding effects of the stimuli from step 305 on one or more neurons and/or neural circuits of the subjects. This data provides information about how the neural activity patterns change in response to different types of stimuli delivered by the stimulatory device. Similar to the first set of training data, each data point in the second set is paired with the stimulus input that elicited the neural response. However, the focus of the second set is on characterizing the observed changes in neural activity rather than the stimuli themselves.

Accordingly, the neural responses elicited by the stimuli designated in step 305 are recorded using imaging devices such as EEG or MEG, resulting in paired data samples consisting of the stimulus input and the corresponding neural activity response from step 310. Both of these datasets are used in tandem to train a machine learning model to predict and analyze neural responses to non-invasive neurostimulation and optimize treatment strategies for individual subjects.

At step 315, the collected data may be annotated and labeled to provide supervision for machine learning algorithms. Supervised learning algorithms rely on annotated data to identify correlations between input features (e.g., stimuli) and output labels (e.g., neural responses), allowing them to generalize from known examples to make predictions on unseen data. By providing labeled examples during training, supervised learning algorithms iteratively adjust their internal parameters to minimize prediction errors and optimize their performance on the given task.

In an aspect, step 315 may involve the manual or automated process of marking specific segments or features within the collected data with labels or annotations. Depending on the nature of the data, annotations may be performed by domain experts, trained annotators, or through automated algorithms designed to detect specific patterns or events in the data. In an aspect, annotating the data may correspond to one or more of: identifying regions of interest in neural activity signals, labeling different types of stimuli presented during neurostimulation sessions, and/or categorizing data according to specific experimental conditions or clinical outcomes.

In an aspect, descriptive labels or categories may be applied to annotated segments of the data based on predefined criteria or classification schemes. These labels provide semantic meaning to the data and facilitate subsequent analysis and interpretation. Labeling schemes may vary depending on the objectives of the study and the types of data being annotated. For example, in neurostimulation research, labels may indicate the presence of abnormal neural activity patterns, the type of stimuli presented (e.g., visual, auditory), or the specific experimental conditions under which the data was collected.

At step 320 feature extraction may be performed to capture relevant characteristics or patterns from neural activity signals that are informative for predicting or analyzing treatment outcomes. In general, feature extraction involves transforming raw data into a format that is suitable for input to machine learning algorithms. Features extracted from neural activity data may encompass various aspects of the signal's characteristics, including temporal, spectral, spatial, and statistical properties. Temporal features describe how neural activity changes over time, such as the amplitude, frequency, and latency of event-related potentials (ERPs) or oscillatory rhythms. Spectral features characterize the frequency content of the neural signal, such as power spectral density, coherence, or phase-locking measures across different frequency bands. Spatial features capture spatial patterns or distributions of neural activity across different brain regions or electrodes, which may reflect functional connectivity networks or localized activation patterns. Statistical features quantify statistical properties of the neural signal, such as mean, variance, or skewness, which provide information about the signal's distribution and dynamics.

Before feature extraction, preprocessing steps may be applied to the raw neural activity data to enhance signal quality and remove artifacts. This may include filtering to remove noise or unwanted frequency components, artifact rejection techniques, baseline correction, and normalization. Preprocessing ensures that the extracted features accurately reflect the underlying neural dynamics and minimize the influence of confounding factors or measurement artifacts. In many cases, neural activity data may be high-dimensional, containing a large number of features or channels. Dimensionality reduction techniques may be applied to reduce the complexity of the data and extract its essential characteristics. For instance, Principal component analysis (PCA), independent component analysis (ICA), or manifold learning methods can be used to identify lower-dimensional representations of the data that preserve its underlying structure and variability. In an aspect, after feature extraction, feature selection techniques may be employed to identify the most informative subset of features for training machine learning models. This helps reduce model complexity, improve generalization performance, and enhance interpretability. Feature selection methods include univariate statistical tests, wrapper methods (e.g., recursive feature elimination), and embedded methods (e.g., L1 regularization), which evaluate the predictive power of individual features or feature subsets.

At step 325, a selected model may be trained to learn patterns and relationships from the training data to make predictions or classifications. In the context of non-invasive neurostimulation, training the model involves using neural activity data and corresponding outcomes to teach the model to accurately predict treatment responses.

In an aspect, one of a variety of different types of model architectures may be selected, depending on the nature of the data. Commonly used models include linear models (e.g., linear regression, logistic regression), decision trees, random forests, support vector machines (SVM), neural networks (e.g., deep learning models), Gaussian processes, and ensemble methods (e.g., boosting, bagging). In non-invasive neurostimulation research, domain-specific knowledge about neurophysiology, neural circuits, and stimulation protocols may inform the selection of models and features.

Before training the selected model, the dataset is typically divided into training, validation, and test sets. The training set is used to teach the model, the validation set is used to tune hyperparameters and monitor performance during training, and the test set is used to evaluate the final performance of the trained model. Data preprocessing techniques, such as normalization, scaling, imputation of missing values, and feature selection, may be applied to prepare the dataset for training.

During training, the model may iteratively process batches of training data and update its parameters to minimize the loss function. In each training iteration (or epoch), the model computes predictions for the input data, calculates the loss between the predicted outputs and the ground truth, and may perform backpropagation to update the model's parameters based on the gradients of the loss function. The training process may continue until a stopping criterion is met, such as reaching a maximum number of epochs or achieving satisfactory performance on the validation set. In an aspect, during training, the model iteratively processes batches of training data and updates its parameters to minimize the loss function. In each training iteration (or epoch), the model computes predictions for the input data, calculates the loss between the predicted outputs and the ground truth, and performs backpropagation to update the model's parameters based on the gradients of the loss function. The training process continues until a stopping criterion is met, such as reaching a maximum number of epochs or achieving satisfactory performance on the validation set.

At step 330, the performance of the trained model may be assessed. More particularly, a cross-validation may be employed to estimate how well a model will perform on unseen data. By systematically partitioning the dataset into training and validation subsets multiple times, cross-validation provides a more robust estimate of the model's performance compared to a single train-test split.

In a non-limiting, exemplary implementation of cross-validation, the dataset may be divided into k folds, with each fold containing approximately equal-sized portions of the data. For example, in 5-fold cross-validation, the dataset is divided into five subsets. The model is trained k times, with each iteration using a different fold as the validation set and the remaining folds as the training set. For instance, in the first iteration, the first fold is used for validation and the remaining folds are used for training. In the second iteration, the second fold is used for validation, and so on. After training the model on each fold, its performance is evaluated on the validation set associated with that fold. Performance metrics such as accuracy, precision, recall, F1 score, or mean squared error are computed for each iteration. The final performance metric is typically computed as the average (or median) of the performance metrics obtained across all k iterations. This provides a more reliable estimate of the model's generalization ability compared to a single train-test split.

At step 335, the fully trained model may be employed in clinical use. More particularly, after successful training and evaluation, the trained model can be deployed in clinical settings to analyze brain activity, predict neural responses to stimuli, or assist in diagnosing medical conditions and designing personalized treatment plans based on individual subject data.

During deployment, electronic and/or magnetic test data may be received comprising data associated with the activity of one or more neural circuits of a subject. This data may be received from an imaging device. In some aspects, a medical condition of the subject may additionally be received. In an aspect, the trained model may identify one or more neural circuits of the subject to target based on the medical condition of the subject and/or the electronic data associated with the activity of the neural circuits of the subject. In some aspects, the subject may be asked to perform motor task(s) and/or cognitive task(s) during data capture, which may help potentiate or target healthy and/or abnormal pathways. The specific motor task and/or cognitive task that the subject is requested to perform may be dynamically identified by the trained model based on the symptoms and/or medical condition of the subject. In an aspect, using a non-invasive stimulatory device, inputs may be provided to stimulate the one or more determined neural circuits (e.g., they identified healthy neural circuits, etc.) of the subject. In an aspect, inputs may be continuously provided with the non-invasive stimulatory device until a predetermined period of time has passed, based on subject feedback, and/or based on feedback from the electronic data associated with activity of one or more determined neural circuits of the subject.

In one technique dynamical maps may be used to inform algorithmic improvements in machine learning models. In this technique, previously recorded visual stimulation and neural data is used to learn a first machine learning model that maps between dynamics in visual stimulation and dynamics in the neural. A second machine learning model searches for visual stimulation in real-time which drives the dynamics in the neural data towards a specific target. The dynamic maps from the first machine learning model are used to make the search task of the second machine learning more efficient.

### Construction and Deployment of a Trained Model for Treating Subjects Having a Scotoma

The data acquisition and machine learning techniques described above may be purposefully directed to address issues involving the presence of a scotoma. A scotoma is an area of partial or complete loss of vision in the visual field. It can be caused by various factors such as damage to the optic nerve or retina, or certain other neurological conditions. Some or all of the steps described herein may be performed in accordance with workflows 200 and 300. In some aspects, scotoma-specific data may be utilized in the training and deployment of the model.

In an aspect, two sets of electronic data may be collected. The first set involves inputs from a non-invasive visual stimulatory device provided to multiple subjects. These inputs may include various visual stimuli such as colors, gradients, shapes, patterns, etc. The second set includes corresponding effects on one or more nerves, including cortical circuits and/or nerves of the retina(s) of the subjects. This data includes the neural responses elicited by the visual stimuli provided in the first set. In some aspects, the second set of training data may include only healthy neural circuits, only data comprising unhealthy/dysfunctional neural circuits, or a combination of the two. Healthy neural circuit training data may be associated with circuits from outside of the central visual field unaffected by the scotoma (e.g., healthy retinal neurons), cortical visual processing circuits, etc. Healthy neural circuits may be associated with or within a predetermined proximity of circuits affected by the scotoma. Unhealthy/dysfunctional neural circuit training data may be associated with the retinal neurons affected by the scotoma or other visual obstruction, or any neural circuitry associated therewith.

Both sets of training data may be annotated to provide supervised training for the machine learning system. This annotation helps the system learn the relationship between the visual stimuli and the neural responses. Specifically, the annotated training data is used to train a machine learning system, potentially employing various machine learning algorithms (e.g., as described above). During clinical use, electronic data associated with the inputs provided by the stimulatory device and/or neural circuit data may be fed back into the machine learning system for retraining purposes. This allows the model to adapt to individual subject responses and improve its effectiveness over time.

During deployment of the trained model in a clinical setting, electronic and/or magnetic data associated with the activity of one or more neural circuits of a subject may be received from an imaging device. This data may contain data associated with the activity of one or more neural circuits of a subject. In some aspects, the data may be received from an imaging device. In some aspects, a medical condition of the subject may be received, which may include the condition, location of any blind spot, affected eye(s), severity, etc.

One or more neural circuits of the subject to target may be determined based on the medical condition of the subject and/or electronic data associated with the activity of the neural circuits of the subject. In some aspects, the neural circuits to target may be healthy neural circuits associated with, or within a predetermined proximity to, abnormal neural circuits. In some aspects, the subject may be asked to perform motor task(s) and/or cognitive task(s) during data capture, which may help potentiate or target healthy and/or abnormal pathways. The motor task and/or cognitive task performed may be determined based on the symptoms and/or medical condition of the subject.

In an aspect, inputs may be provided to the subject (e.g., using a non-invasive stimulatory device, etc.) to visually stimulate the one or more neural circuits of the subject. In some aspects, a specific, or predetermined, neural circuit may be targeted for stimulation based on the indicated medical condition of the subject. This neural circuit may be limited to healthy neural circuits, for example healthy retinal neurons.

The non-invasive stimulatory device may continue to provide inputs until a predetermined period of time has passed or until a predetermined event is detected (e.g., specific feedback from the subject is received, specific feedback from the electronic data associated with activity of one or more predetermined neural circuits of the subject is received, etc.). In some aspects, the subject may be instructed to perform a task while inputs from the non-invasive stimulatory device are being shown. For instance, the subject may be asked to repeatedly perform a motor or cognitive task, such as picking up an object or thinking about picking up an object. As another example, the subject may be asked to identify and/or focus on the object. In an aspect, a biofeedback loop may be used to optimize therapy, as discussed above with respect to workflow 200.

### Construction and Deployment of a Trained Model for Treating Subjects Experiencing Phantom Limb Syndrome

The data acquisition and machine learning techniques described above may be purposefully directed to address issues involving phantom limb syndrome. Phantom limb syndrome is a condition in which subjects experience sensations, whether painful or otherwise, in a limb that does not exist. Such a condition is often found in amputees. Some or all of the steps described herein may be performed in accordance with workflows 200 and 300. In some aspects, data specific to phantom limb syndrome may be utilized in the training and deployment of the model.

In an aspect, two sets of electronic or magnetic training data may be received. The first training set may encompass inputs provided by a non-invasive visual stimulatory device to multiple subjects. These inputs may include provision of a color, gradient, shape, picture, edge, object rotation, pattern, one or more flashes at predetermined time intervals and/or wavelengths, etc., to the subject. In some aspects, the first set may also include data indicating a medical condition, if any, of the associated subject. The medical condition may include missing limb/digit, and/or symptoms such as pain, itching, clenching, hot, and/or cold in the phantom limb. In an aspect, the subjects may be required to perform motor and/or cognitive tasks during data capture, aiding in targeting healthy or abnormal pathways based on symptoms or medical conditions.

In an aspect, the second training set may encompass corresponding effects on nerves and/or neural circuits, potentially relating to cortical circuitry, nerve operations in the peripheral nervous system, and human extremities/digits. The second training set may include only healthy neural circuits, only data comprising unhealthy/dysfunctional neural circuits, or a combination of the two. Healthy neural circuit training data may be associated with or otherwise within a predetermined pathway of abnormal neural circuits. The pathway may consist of processing centers that are subcortical and/or cortical. In this way, stimulus of normal neural pathways, for example through the eye, may act to stimulate a healthy processing center in the brain in proximity to an abnormal processing center in the brain associated with the phantom limb dysfunction.

Both sets of training data may be annotated to facilitate supervised training of a machine learning system. Electronic data associated with stimulatory device inputs and neural circuit data may be provided back to the machine learning system for retraining purposes.

During deployment of the trained model in a clinical setting, electronic and/or magnetic data associated with the activity of one or more neural circuits of a subject may be received from an imaging device. This data may contain data associated with the activity of one or more neural circuits of a subject. In some aspects, a medical condition of the subject may be received, which may include an indication of a missing limb/digit, and/or symptoms such as pain, itching, clenching, hot, and/or cold in the phantom limb/digit.

Based on the subject's medical condition and electronic data associated with neural activity, one or more neural circuits to target may be determined. These target circuits may include healthy circuits associated with or in proximity to abnormal circuits related to phantom limb symptoms. In some aspects, the neural circuits to target may be healthy neural circuits associated with, or within a predetermined proximity to, abnormal neural circuits. In some aspects, the subject may be asked to perform motor task(s) and/or cognitive task(s) during data capture, which may help potentiate or target healthy and/or abnormal pathways. The motor task and/or cognitive task performed may be determined based on the symptoms and/or medical condition of the subject.

In an aspect, inputs may be provided to the subject (e.g., using a non-invasive stimulatory device, etc.) to visually stimulate the one or more neural circuits of the subject. In some aspects, a specific, or predetermined, neural circuit may be targeted for stimulation based on the indicated medical condition of the subject. This neural circuit may be limited to healthy neural circuits, for example healthy cortical circuits associated with afferent neurons of the missing limb, or adjacent or related circuits such as visual stimulation to interrupt the thalamic and ganglia-based signal processing, and/or cortical interpretation of pain. Limb sensory, eye sensory, or both may also be stimulated.

The non-invasive stimulatory device may continue to provide inputs until a predetermined period of time has passed or until a predetermined event is detected (e.g., specific feedback from the subject is received, specific feedback from the electronic data associated with activity of one or more predetermined neural circuits of the subject is received, etc.). In some aspects, the subject may be instructed to perform a task while inputs from the non-invasive stimulatory device are being shown. For instance, the subject may be asked to repeatedly perform a motor or cognitive task, such as picking up an object or thinking about picking up an object. As another example, the subject may be asked to identify and/or focus on the object. In an aspect, a biofeedback loop may be used to optimize therapy, as discussed above with respect to workflow 200.

The functional modules described herein are provided by way of example. It will be understood that different functional modules may be combined to create different utilities. It will also be understood that additional functional modules or sub-modules may be created to implement a certain utility.

In general, any process discussed in this disclosure that is understood to be computer-implementable may be performed by one or more processors of a computer system, such as system environment 110, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer server. A processor may be a central processing unit (CPU), a graphics processing unit (GPU), or any suitable types of processing unit.

A computer system, such as system environment 110, may include one or more computing devices. If the one or more processors of the computer system are implemented as a plurality of processors, the plurality of processors may be included in a single computing device or distributed among a plurality of computing devices. If a system environment comprises a plurality of computing devices, the memory of the computer system may include the respective memory of each computing device of the plurality of computing devices.

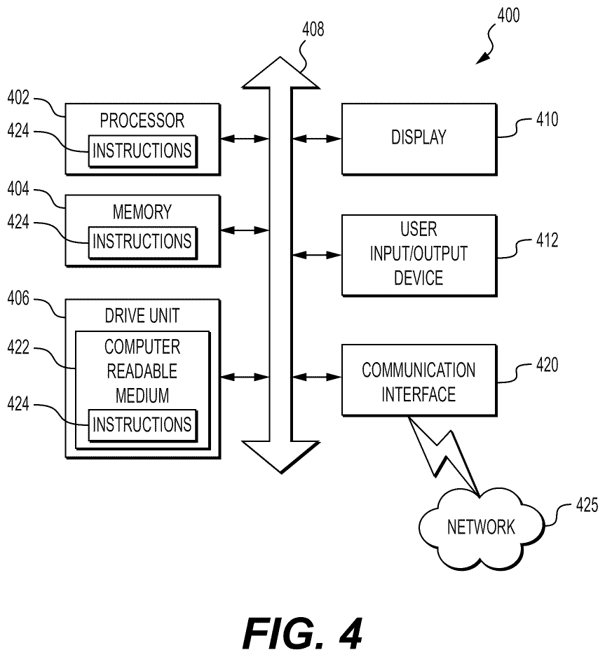

FIG. 4 is a simplified functional block diagram of a computer system 400 that may be configured as a computing device for executing the processes described herein, according to exemplary embodiments of the present disclosure. FIG. 4 is a simplified functional block diagram of a computer that may be configured according to exemplary embodiments of the present disclosure. In various embodiments, any of the systems herein may be an assembly of hardware including, for example, a data communication interface 420 for packet data communication. The platform also may include a central processing unit (“CPU”) 402, in the form of one or more processors, for executing program instructions. The platform may include an internal communication bus 408, and a storage unit 406 (such as ROM, HDD, SDD, etc.) that may store data on a computer readable medium 422, although the system 400 may receive programming and data via network communications via electronic network 425, which may correspond to network 40 (e.g., voice, video, audio, images, or any other data over the electronic network 425). The system 400 may also have a memory 404 (such as RAM) storing instructions 424 for executing techniques presented herein, although the instructions 424 may be stored temporarily or permanently within other modules of system 400 (e.g., processor 402 and/or computer readable medium 422). The system 400 also may include input and output ports 412 and/or a display 410 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. Relative terms, such as “about,” “approximately,” “substantially,” and “generally,” are used to indicate a possible variation of ±10% of a stated or understood value. In addition, the term “between” used in describing ranges of values is intended to include the minimum and maximum values described herein. The use of the term “or” in the claims and specification is used to mean “and/or” unless explicitly indicated to refer to alternatives only if the alternatives are mutually exclusive, although the disclosure supports a definition that refers to only alternatives and “and/or.” As used herein “another” may mean at least a second or more.

As used herein, the term “user” generally encompasses any person or entity, such as a researcher and/or a care provider (e.g., a doctor, etc.), that may desire information, resolution of an issue, or engage in any other type of interaction with a provider of the systems and methods described herein (e.g., via an application interface resident on their electronic device, etc.). The term “electronic application” or “application” may be used interchangeably with other terms like “program,” or the like, and generally encompasses software that is configured to interact with, modify, override, supplement, or operate in conjunction with other software.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device.

Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A computer-implemented method for modulating neural pathways to improve functional ability in a subject, the computer-implemented method comprising:
receiving, at a computing device, a first dataset associated with activity of one or more neural pathways of the subject;
receiving, at the computing device, a second dataset comprising at least one of at least one task performed by the subject, at least one symptom, and at least one medical condition of the subject;
determining, using a processor associated with the computing device, one or more abnormal neural pathways based on the first dataset and the second dataset;
determining, using the processor and from the second dataset, one or more healthy neural pathways associated with the one or more abnormal neural pathways based on one or more criteria; and
determining, based on the healthy neural pathways and abnormal neural pathways, and using a non-invasive stimulatory device, a stimulus input to the healthy neural pathways.

2. The computer-implemented method of claim 1, wherein the first dataset encompasses at least one signal associated with at least one of: electrical activity, magnetic activity, and/or blood flow activity.

3. The computer-implemented method of claim 1, wherein the determining the one or more abnormal neural pathways comprises comparing the first dataset to reference data associated with predetermined normal brain activity.

4. The computer-implemented method of claim 1, wherein the tasks include motor tasks and/or cognitive tasks.

5. The computer-implemented method of claim 1, wherein a selection of a type of the tasks is determined by a trained machine learning model.

6. The computer-implemented method of claim 1, wherein the one or more criteria include a proximity of the one or more healthy neural pathways to the one or more abnormal neural pathways.

7. The computer-implemented method of claim 1, further comprising:
applying the stimulus input, to the subject, at a predetermined frequency.

8. The computer-implemented method of claim 7, wherein the applying comprises applying the stimulus input until a predetermined period of time has passed or until a predetermined event is detected.

9. The computer-implemented method of claim 1, further comprising:
determining whether the stimulus input improved neural activity and/or improved functional ability in the subject; and
upon determining that the stimulus input did not improve the neural activity and/or functional ability in the subject beyond a predetermined threshold, applying at least one additional type of stimulus input to the one or more healthy neural pathways.

10. The computer-implemented method of claim 1, further comprising implementing a biofeedback loop, wherein the implementing comprises:
receiving biofeedback data from the subject receiving the stimulus input;
applying the biofeedback data to a trained machine learning model; and
receiving output from the trained machine learning model corresponding to a second stimulus input and/or course of treatment for the subject.

11. A system for modulating neural pathways to improve functional ability in a subject, the system comprising:
one or more processors;
one or more computer readable media storing instructions that are executable by the one or more processors to perform operations comprising:
receiving, at a computing device associated with the system, a first dataset associated with activity of one or more neural pathways of the subject;
identifying one or more abnormal neural pathways based on the first dataset;
receiving, at the computing device, a second dataset associated with tasks performed by the subject;
determining, from the second dataset, one or more healthy neural pathways associated with the one or more abnormal neural pathways based on one or more criteria; and
applying, based on the determining and using a non-invasive stimulatory device associated with the system, a stimulus input to the one or more healthy neural pathways.

12. The system of claim 11, wherein the first dataset encompasses at least one signal associated with at least one of: electrical activity, magnetic activity, and/or blood flow activity.

13. The system of claim 11, wherein the determining the one or more abnormal neural pathways comprises comparing the first dataset to reference data associated with predetermined normal brain activity.

14. The system of claim 11, wherein a selection of a type of the tasks is determined by a trained machine learning model.

15. The system of claim 11, wherein the one or more criteria include a proximity of the one or more healthy neural pathways to the one or more abnormal neural pathways.

16. The system of claim 11, the operations further comprising:
applying the stimulus input, to the subject, at a predetermined frequency.

17. The system of claim 16, wherein the applying comprises applying the stimulus input until a predetermined period of time has passed or until a predetermined event is detected.

18. The system of claim 11, the operations further comprising:
determining whether the stimulus input improved neural activity and/or improved functional ability in the subject; and
upon determining that the stimulus input did not improve the neural activity and/or functional ability in the subject beyond a predetermined threshold, applying at least one additional type of stimulus input to the one or more healthy neural pathways.

19. The system of claim 11, further comprising implementing a biofeedback loop, wherein the implementing comprises:
receiving biofeedback data from the subject receiving the stimulus input;
applying the biofeedback data to a trained machine learning model; and
receiving output from the trained machine learning model corresponding to a second stimulus input and/or course of treatment for the subject.

20. A non-transitory computer-readable medium storing computer-executable instructions which, when executed by a system, cause the system to perform operations comprising:
receiving, at a computing device, a first dataset associated with activity of one or more neural pathways of a subject;
identifying, using a processor associated with the computing device, one or more abnormal neural pathways based on the first dataset;
receiving, at the computing device, a second dataset associated with tasks performed by the subject;
determining, using the processor and from the second dataset, one or more healthy neural pathways associated with the one or more abnormal neural pathways based on one or more criteria; and
applying, based on the determining and using a non-invasive stimulatory device, a stimulus input to the one or more healthy neural pathways.
